(19) United States
(12) Patent Application Publication
(10) Pub. No.: US 2003/0198695 A1
(43) Pub. Date: Oct. 23, 2003

(54) Herbo-mineral composition

(75) Inventor: Shibnath Ghosal, Varanasi (IN)

Correspondence Address: DR. WALTER KATZ, 8 UMBERLAND PLACE, MONROE TOWNSHIP, NJ, 08831, US

(73) Assignees: NATREON INC.; INDIAN HERBS RESEARCH; SUPPLY COMPANY LTD.

(21) Appl. No.: 10/128,832

(22) Filed: Apr. 23, 2002

(51) Int. Cl. A61K 35/78; A61K 33/24

(52) U.S. Cl. 424/725; 424/617

(57) ABSTRACT

A herbo-mineral composition includes a mineral-complexing agent which is purified Shilajit containing dimeric and/or oligomeric dibenzo-α-pyrones (DBPs), and, optionally, in synergistic combination with an extract of Emblica officinalis containing gallo/ellagi-tannoids (GET), and, one or more added minerals, such as iron, copper or chromium. The composition in the example where the added mineral is iron is particularly effective in treating iron-deficiency anemia by rapidly absorbing ferrous iron into the blood stream without side effects.

### CROSS-REFERENCE TO RELATED PATENTS AND PATENT APPLICATIONS

This application is related to U.S. Pat. No. 6,124,688, and to U.S. patent application Ser. No. 09/860,890, filed May 18, 2001, and Ser. No. 09/957,797, filed Sep. 21, 2001, which are incorporated by reference herein.

### BACKGROUND OF THE INVENTION

1. Field of the Invention

This invention relates to herbo-mineral compositions for treating mineral-deficient conditions, and, more particularly, to compositions which include a bioactive metal-complexing agent which is purified Shilajit containing purified dimeric and oligomeric dibenzo-α-pyrones (DBPs), obtained from native Shilajit, and, optionally, in synergistic combination with gallo/ellagi tannoids (GET) extracted from the Emblica officinalis plant, and an added mineral supplement, such as iron, copper, chromium and the like, which compositions can be readily absorbed in the body without gastric upset or side effects.

2. Description of the Prior Art

Anemia is defined as a deficiency of erythrocytes or hemoglobin per unit volume of blood. Its clinical manifestations are pallor of mucous membrane, dullness and listlessness due to relative tissue hypoxia. In its later stages, anemia is manifested by an increase in the rate and force of the heart beat, and extreme weakness. These symptoms also are associated with a decreased ability to fight infections, or to cope with any kind of stress, for example, a pregnancy, lactation in females, or normal growth in children.

The most common anemias are deficiency anemias arising from lack of iron, folic acid and/or cyanocobalamine. In developing countries, it is iron deficiency that results in most of the clinically observed cases of anemia. This form of deficiency often arises from parasitic infestation, particularly in children, an increased demand and limited iron supply from repeated pregnancies, or chronic blood loss with menstrual irregularities like hemorrhagic. Thus a higher incidence of iron deficiency anemia is observed in the female population and in growing children. For proper treatment, it is necessary to provide iron in therapeutic doses in a form that is easily absorbed by the body without side effects.

An average human male has about 4.5 g of iron in its body. Normally, about 1 mg of iron is absorbed from his diet daily, and the same amount is excreted during the same period. Since the total plasma iron turnover per day is about thirty times that of the iron absorption ratio, an extremely efficient mechanism for maintenance of iron balance must exist in the body. Even a slight disturbance of iron metabolism leads to iron-deficiency or an iron-overload, both being detrimental to health.

There are two kinds of iron in the diet which affect the mechanism of iron absorption, namely, haem-iron and non-haem iron, each utilizing separate receptors on the mucosal cells. After the uptake of haem-iron into the mucosal cells, its porphyrin ring is split by a special enzyme (haemoxygenase) within the cells and its iron is released. For non-haem iron, receptors on the luminal side compete with complexing luminal ligands for the iron ions; accordingly, its iron can only be absorbed into the cells and pass the mucosal membrane in the ferrous form. Therefore reducing substances must be present in the mucin layer of the mucosal cells for ferrous iron to be absorbed.

About two-thirds of iron in the body is found in haemoglobin, the rest in myoglobin, various other iron-containing enzymes and iron-transport proteins e.g., transferrin. Excess iron is stored as ferritin and haemosiderin, the latter being an insoluble product which occasionally is involved in the systemically deleterious free radical reaction known as the Fenton reaction.

When an iron over-dose occurs, or even a slight disturbance in iron metabolism, a Post-Fenton (Haber-Weiss) reaction can generate hydroxyl radicals with concomitant breaking of DNA strands, activation of poly-ADP ribose synthetase and NAD depletion, resulting in a marked reduction in cellular energy. These deleterious effects, which are consequences of the body's inability to dispose of excess iron, are manifested as gastro-intestinal bleeding and peripheral vascular collapse. Such symptoms also are found when high doses of inorganic iron compounds are taken which are not present in the form of iron complexes and/or iron chelates.

There are many expensive iron preparations available in the market today for the prevention and treatment of anemia. However, their effectiveness is uncertain, or associated with side-effects, and many need to be taken by injection. For example, parentral iron therapy is painful and often associated with development of anaphylaxis or late serum sickness; hence it is not suitable for many anemia patients. Oral iron therapy, on the other hand, requires heavy doses of ferrous salts, and may cause gastrointestinal distress, abdominal pain, nausea, vomiting, constipation or diarrhea, often in more than 25% of patients. Since iron therapy needs to be continued for at least 6 months, iron-supplementation with commonly available preparations often is required to rebuild reticulo-endothelial iron content.

Other mineral-deficient conditions in the body are known, which stem from an imbalance of, for example, chromium, copper, zinc, manganese and/or other elemental minerals. The present invention also addresses these conditions.

Accordingly, it is an object of this invention to provide a novel herbo-mineral composition containing predetermined low levels of added minerals, e.g. iron, suitably in the form of capsules or syrup, which can be readily absorbed by the body when given orally, and without gastric upset, and which has a simple, daily dosing schedule.

A feature of the invention is the provision of a herbo-mineral composition which includes a bioactive herbo-mineral complexing agent which is purified Shilajit containing dimeric and/or oligomeric dibenzo-α-pyrones (DBPs), optionally, but preferably, with an extract of Emblica officinalis containing gallo/ellagi tannoids (GET), and an added mineral supplement.

These and other objects and features of the invention will be made apparent from the following description.

### SUMMARY OF THE INVENTION

What is described herein is a herbo-mineral composition for the treatment or prevention of mineral-deficient conditions which includes purified Shilajit containing dimeric and/or oligomeric dibenzo-α-pyrones (DBPs), optionally, but preferably, in synergistic combination with an extract of the Emblica officinalis plant containing gallo/ellagi-tannoids (GET), and an added mineral supplement. The DBPs (and GETs) are the primary bioactive constituents in the composition. These complexing agents exert their natural reducing activity on the added mineral, e.g. iron, chromium, copper, zinc, manganese and the like, to maintain it in a bioavailable and effective oxidation state.

The DBPs in the composition suitably have a molecular weight ranging from about 450 to 2500 Daltons, preferably 500-700 Daltons, which allows for ready absorption of the metal ion-DBP complex from a fine, aqueous colloidal suspension of the composition.

### DETAILED DESCRIPTION OF THE INVENTION

The composition of the invention is a herbo-mineral formulation useful for the treatment and prevention of mineral-deficient conditions e.g. anemia, in both children and adults. The composition is an admixture of dimeric and/or oligomeric dibenzo-α-pyrones (DBPs), e.g. as metallic complexes, isolated from purified and processed Shilajit*, and an added mineral, e.g. iron, chromium, copper, zinc, manganese, and the like. The composition preferably also includes a herbal extract of Emblica officinalis** which contains small molecular weight gallo-ellagi tannoids (GET). When present, the GET herbal extract synergistically enhances the coordinating site of the DBP-metal complex to maintain the complex in its reduced state, e.g. iron in the ferrous state, which can be more effectively utilized in the synthesis of hemoglobin. * U.S. patent application Ser. No. 09/860,890, filed May 18, 2001, incorporated by reference herein. ** U.S. Pat. No. 6,124,688, incorporated by reference herein.

In the past, when iron in the ferrous form was given without complexation, it was rapidly converted into ferric iron in the body resulting in poor iron absorption as well as formation of toxic free radicals. In the complexed form in the composition of this invention, ferrous iron is not converted into ferric form because ferrous iron is complexed with the DBPs (and GETs) which become trapped and stabilized in the micropores (d=100-150 A) of the purified Shilajit.

The dimeric and oligomeric DBP-humato-ferrate (Fe²⁺) complex in the composition herein suitably has a low molecular weight of about 450-2500 Daltons, preferably 500-700 Daltons, commensurate with rapid absorption of the added mineral in the body.

The following Table illustrates the defined composition of the invention:

TABLE

| | Suitable | Preferred |
|---|---|---|
| Ingredient | Amount (mg) | Amount (mg) |
| Purified Shilajit | 5-200 | 10-25 |
| Emblica officinalis extract | 0-500 | 50-150 |
| Added Mineral Supplement | 0.1-250 | 5-25 |

A typical composition of the invention, in the form of 100 ml of syrup, for example, includes about 200 mg of purified Shilajit, about 500 mg of Emblica officinalis extract, and about 250 mg of added mineral, e.g. Natural Product, which contains w/w 35-40% Fe and 15-18% Cu, and excipients, to q.s. The resultant formulation is a viscous, pourable liquid, dark-brown in color, with a pleasant odor, and a sweet-to-mildly bitter taste. The composition has a density of about 1.2 g/ml, a pH of 4.4, and a solids content (w/w) of 68%.

Another typical formulation of the invention composition is a 100 mg powder which contains 2.76 mg of elemental iron supplement, in a highly bioavailable complexed form which can be easily administered orally.

The following examples will illustrate the invention, more particularly, with reference to compositions of the invention where the added mineral is iron.

### EXAMPLE 1

### IRON PREPARATION CAPSULE

| Ingredient | Quantity per capsule (mg) |
|---|---|
| Purified Shilajit | 25 |
| Emblica officinalis | 50 |
| Elemental Iron | 10 |
| Excipients | q.s. |

### EXAMPLE 2

### IRON PREPARATION SYRUP

| Ingredient | Quantity per 100 ml (mg) |
|---|---|
| Purified Shilajit | 250 |
| Emblica officinalis | 500 |
| Elemental Iron | 100 |
| Excipients | q.s. |

The results of a clinical evaluation of the efficacy and tolerability of iron preparations for iron-deficiency anemia is illustrated by the data below:

BIO-CHEMICAL EFFICACY PARAMETER PROFILE OF STUDY GROUPS-TEST &
REFERENCE PRODUCTS
Test Product: Iron Preparation in Capsule form where a Natural Product
was used as the source of elemental iron
Reference Product: Allopathic drug marketed by a Multi-National
Pharmaceutical company

| | | No. of | | | | |
|---|---|---|---|---|---|---|
| | | Patients | | | | |
| | | With | | | | 30 days |
| | | Abnormal | No. of | % of | Baseline | Study end |
| Parameter | | Values at | Patients | Patients | Value | Value |
| [Abnormal,if] | Product | Baseline | Improved | Improved | [Mean ± SEM] | [Mean ± SEM] |
| Haemoglobin (g/dl) | Test | 15/15 | 10/15 | 66.67 | 9.69 ± 0.273 | 12.67 ± 0.361*** |
| | Reference | 16/16 | 3/16 | 18.75 | 10.58 ± 0.264 | 11.94 ± 0.189** |
| ]<13 g/dl (in Males) | | | | | | |
| <12 g/dl (in Females)] | | | | | | |
| Hamatocrit (%) | Test | 14/14 | 9/14 | 64.28 | 31.10 ± 0.86 | 39.30 ± 1.08*** |
| | Reference | 15/16 | 5/15 | 33.33 | 33.30 ± 0.86 | 36.90 ± 0.87* |
| [47 ± 5% (in Males) | | | | | | |
| 42 ± 5% (in Females)] | | | | | | |
| Serum Iron (mcg/dl) | Test | 4/14 | 4/4 | 100 | 66.40 ± 6.91 | 90.90 ± 6.74** |
| | Reference | 5/16 | 4/5 | 80 | 88.50 ± 11.23 | 92.50 ± 5.53 |
| [50-150 mcg/dl] | | | | | | |

| Parameter | | Improvement | % Improvement | Avg. improvement |
|---|---|---|---|---|
| [Abnormal,if] | Product | from Baseline | from Baseline | per week |
| Haemoglobin (g/dl) | Test | +2.98 | 30.73 | 0.75 gm/dl |
| | Reference | +1.36 | 12.85 | 0.34 gm/dl |
| [<13 g/dl (in Males) | | | | |
| <12 g/dl (in Females)] | | | | |
| Hamatocrit (%) | Test | +8.20 | 26.37 | 2.05% |
| | Reference | +3.60 | 10.81 | 0.90% |
| [47 ± 5% (in Males) | | | | |
| 42 ± 5% (in Females)] | | | | |
| Serum Iron (mcg/dl) | Test | +24.50 | 36.90 | 6.13 mcg/dl |
| | Reference | +4.00 | 4.52 | 1.00 mcg/dl |
| [50-150 mcg/dl] | | | | |

Abbreviations:
SEM-Standard Error of Mean
mcg-Microgram
*2-tailed p < 0.05 (paired t-test) in comparison to baseline
**2-tailed p < 0.005 (paired t-test) in comparison to baseline
***2-tailed p < 0.001 (paired t-test) in comparison to baseline

Since slow release and re-absorption of the metal ion are inherent properties of the DBP-metal complex, the composition of the invention does not suffer from the limitations of prior compositions, e.g. amino acid-mineral chelates. These compositions exhibit metal ion irreversibility, which leave the metal ion in the undesirable higher oxidation state. For example, in iron-amino acid chelates, two of the six coordination sites of iron are unfilled by aqua-ligands, which increases the possibility of oxidation of ferrous to ferric iron. By contrast, in the present iron-DBP complex, all the coordination sites of ferrous iron are occupied; hence, ferrous iron therein is not readily oxidizable to ferric iron.

Moreover, while iron in the ferrous state in amino-acid chelates can experience difficulty in absorption and release of its iron, iron in the complexed ferrous form in the composition of this invention is released smoothly and absorbed readily into iron-deficient cells. In such an iron-complex, the molar ratio of ligand-to-iron is higher than that of the metal ion; hence, free iron which is not absorbed is again complexed with ligand(s) to prevent a Fenton reaction and its resultant free radical induced damage. Thus, the invention composition assures maximum absorption of iron without toxicity problems.

The iron-organo complex of the invention also is observed to behave similarly to transferrin because its iron can enter various cells of the body to synthesize iron-containing enzymes and proteins. Receptor-mediated endocytosis then can present the transferrin-like iron entity to the iron-deficient cells where it can enter the cytoplasm in a vacuole. At an acidic pH, the contents of the vacuole then can release iron from the iron-chelate with various cellular constituents, e.g. citrate, ATP and GTP. The iron-free assembly is then ejected from the cells for recapture of iron from an iron-free pool.

As a fine colloidal suspension with water, the composition of the invention can act as a potent and tenacious scavenger of both transition and fixed valency metal ions and thus it is capable of penetrating into different cell types. The invention complex, moreover, does not degrade in the presence of free radicals; instead, it can interact with free radicals to generate additional complexing and chelating entities (e.g. during interaction with an iron overload), which can further sequester toxic metal ions. Thus, in small amounts, the invention composition is capable of handling a large amount of iron overload to modulate the iron content in the body with notable beneficial results.

The DBP-polyphenol moieties present in the complex of the invention also is effective in reducing Fe³⁺ to Fe²⁺; thus it will systemically mobilize and funtionalize systemic iron to hemoglobin. These moieties can abstract Fe³⁺ from free metal ions to form readily absorbable Fe²⁺ complexes, and to present and slowly release the metal ion to metal-deficient receptors.

In summary, the composition of the invention is an effective herbo-mineral formulation for prevention and treatment of several forms of anemia caused by hemorrhage, parasitic infestation, nutritional deficiencies, etc. in both children and adults, and in quickly raising the hemoglobin level of blood, e.g. by 1 gm/dl or more per week, without side effects. It also is especially useful for persons with mal-absorption syndromes, those suffering from chronic inflammatory disease, for people who have been taking NSAIDs for long periods, and for individuals suffering from inefficient production of erythrocytes, or increased loss of RBC in hemorrhagic anemia, and also for persons who normally do not respond to conventional chemical metal preparations.

While the invention has been illustrated with particular emphasis on iron as the added mineral supplement, it will be understood that other minerals needed as a nutritional or medicinal supplement may be included as well in the composition of the invention, for example, chromium, copper, zinc, manganese, and the like. Also changes and modifications of the embodiments herein may be made which are equivalent thereto and within the skill of the art. Accordingly, it is intended to be bound only by the following claims, in which:

What is claimed is:

1. A herbo-mineral composition suitable for the treatment of metal-deficient conditions, comprising purified Shilajit containing dimeric and/or oligomeric dibenzo-α-pyrones (DBPs), optionally, in synergistic admixture with an extract of the Emblica officinalis plant containing gallo-ellagi tannoids (GET), and one or more added minerals which form a metal-ion complex with said DBP.

2. A herbo-mineral composition according to claim 1 wherein the added mineral is iron, chromium, copper, zinc, or manganese, or mixtures thereof.

3. A herbo-mineral complex according to claim 2 wherein said added mineral is iron.

4. A herbo-mineral composition according to claim 1 wherein said DBPs have a molecular weight of about 450 to 2500 Daltons.

5. A herbo-mineral composition according to claim 4 wherein said molecular weight is about 500 to 700 Daltons.

6. A herbo-mineral composition according to claim 1 wherein both an purified Shilajit and an extract of the Emblica officinalis plant are present in said composition.

7. A herbo-mineral composition according to claim 1 comprising about 5-200 mg of purified Shilajit, 0-500 mg of an extract of the Emblica officinalis plant, and about 0.1-250 mg of an added mineral supplement.

8. A herbo-mineral composition according to claim 7 wherein both said purified Shilajit and said extract of Emblica officinalis plant are present therein.

9. A herbo-mineral composition according to claim 8 comprising about 10-25 mg of said purified Shilajit, 50-150 mg of said Emblica officinalis extract, and about 5-25 mg of said added mineral supplement.

10. A formulation for the treatment of anemia which includes the composition of claim 1.

11. A formulation according to claim 10 wherein said added mineral is iron, chromium, copper, zinc or manganese.

12. A formulation according to claim 11 wherein said added mineral is iron.
